(19) United States
(12) Patent Application Publication
(10) Pub. No.: US 2004/0148014 A1
(43) Pub. Date: Jul. 29, 2004

(54) Drawn expanded stent

(76) Inventors: Juha-Pekka Nuutinen, Tampa, FL (US); Pertti Tormala, Tampere (FI); Claude O. Clerc, Marlborough, MA (US)

Correspondence Address: SYNNESTVEDT & LECHNER, LLP, 2600 ARAMARK TOWER, 1101 MARKET STREET, PHILADELPHIA, PA, 191072950

(21) Appl. No.: 10/663,507

(22) Filed: Sep. 15, 2003

Related U.S. Application Data
(60) Provisional application No. 60/410,687, filed on Sep. 13, 2002.

(51) Int. Cl. A61F 2/06

(52) U.S. Cl. 623/1.15

(57) ABSTRACT

A polymer stent is disclosed for treating vascular disorders. The stent is formed from a billet having regions with varying plastic strain imposed by drawing the regions through different sized dies at different speeds under different loads. The drawing orients the molecules of the polymer lengthwise along the stent. The stent is placed on a catheter and positioned within the vascular vessel being treated. The stent is expanded radially outwardly by a balloon on the catheter, expansion being facilitated by heat applied to the stent from the balloon. The polymer molecules are oriented circumferentially by the expansion and the mechanical properties of the stent are established. The billet has different compounds, such as medicaments and radiopaque markers distributed throughout the regions. The medicaments are released into the blood stream. The stent may be bio-stable or bio-absorbable.

### RELATED APPLICATION

This application is based on and claims priority to U.S. Provisional Application No. 60/410,687, filed Sep. 13, 2002.

### FIELD OF THE INVENTION

This invention relates to stents for the treatment of vascular stenoses and especially to stents formed of drawn and expanded polymer materials.

### BACKGROUND OF THE INVENTION

Cardiovascular disorders involving stenosis of coronary arteries are increasingly being treated effectively by angioplasty techniques that are less invasive than procedures such as bypass operations. Angioplasty involves dilating a blood vessel, narrowed or occluded by the accumulation of plaque, through the use of a balloon catheter. The catheter is inserted percutaneously through the lumen of the vessel to position the balloon at the site of the narrowing. The balloon on the catheter is inflated to flatten the plaque against the vessel wall and thereby open the vessel to its normal diameter.

After the vessel is expanded by the angioplasty procedure, it may be held in the expanded position by a stent that is implanted within the vessel. The stent acts as a support, providing an outwardly directed radial force that maintains the patency of the vessel. The stent must have adequate strength and stiffness so as to maintain its shape and keep the vessel open. The stent must also be flexible and compliant so as to accommodate movement of the surrounding tissue. It is often desired to use the stent to deliver medicaments to the vascular system, such as anti-thrombogenic drugs that facilitate the treatment and help ensure success of the procedure, for example, by preventing or mitigating the formation of blood clots which could cause a stroke.

In view of these competing requirements, the design of coronary stents for the treatment of stenoses often involves a difficult tradeoff of material and geometric properties in order to obtain the required characteristics such as strength, stiffness and porosity that allow the stent to efficiently and effectively perform its function. For example, stent designs which maximize the strength or stiffness for a given stent geometry and material may not be effective at delivering medicaments of the proper dosage or at the proper dosage rate. Similarly, stents that are capable of carrying significant amounts of medicaments and of releasing the medicaments into the blood stream may not have the strength or stiffness properties to adequately support and maintain vessel patency. There is clearly a need for a stent that has the combination of characteristics that allow it to perform all of its design goals without significantly compromising the effectiveness of any of these intended functions.

### SUMMARY OF THE INVENTION

The invention concerns a stent implantable within a vessel to support the vessel and ensure patency thereof, for example to treat a coronary stenosis. The stent comprises an elongated billet of polymer material, preferably bio-absorbable material. The billet has one or more regions, and each region is pre-drawn lengthwise and has a respective predetermined degree of lengthwise plastic strain imparted by the draw. A lumen extends lengthwise through the billet, and the billet is expanded radially outwardly from the lumen. The regions may be positioned next to one another lengthwise along the billet or overlying one another surrounding the lumen. The region or regions have a respective predetermined degree of circumferential plastic strain imparted by the outward expansion.

Preferably, the billet is heated before and during the radial expansion to a temperature above the glass transition temperature of the polymer. The radial expansion is effected after the billet is positioned within the vessel. This allows the billet to initially assume a small size, allowing it to be delivered percutaneously via a catheter, and then be expanded to fit the vessel that is being treated.

The lengthwise draw orients the molecules comprising the polymer in a direction lengthwise along the billet in response to the lengthwise plastic strain imposed by the draw. Similarly, the molecules comprising the polymer are oriented circumferentially around the lumen in response to the circumferential plastic strain imparted by the expansion. By controlling the degree of draw and expansion, the mechanical properties of the stent, such as strength, stiffness and porosity can be established.

The billet may comprise a molded body or may be formed from a plurality of interlaced filamentary members. The filamentary members may be interlaced by weaving, braiding or knitting.

A compound may be distributed throughout the polymer material in one or more regions of the billet. The compound may be a medicament to be delivered by elutation from the billet or it may be a radiopaque marker to allow viewing of the position of the stent by fluoroscopic techniques. The draws and expansions can be tailored to substantially maximize mechanical strength of the stent, as well as to substantially maximize release of medicament from the stent.

The invention includes a method of making a stent implantable within a vessel to support the vessel and ensure its patency. The method comprises the steps of:

(A) supplying an elongated billet formed of a polymer material;

(B) drawing the billet lengthwise to establish a predetermined degree of lengthwise plastic strain; and

(C) forming a lumen extending lengthwise along the billet.

The method may further include the step of expanding the billet radially outwardly from the lumen to establish a predetermined degree of circumferential plastic strain therein. The expansion step is facilitated by a heating step wherein the billet is heated to a temperature, preferably above the glass transition temperature but below the melting temperature, before and during the radial expansion step.

The drawing step may further comprise the steps of drawing a first region of the billet to a first predetermined degree of the lengthwise plastic strain, and drawing a second region of the billet to a second predetermined degree of the lengthwise plastic strain, the second predetermined degree of the lengthwise plastic strain being different from the first predetermined degree of lengthwise plastic strain.

Furthermore, the expanding step may further comprise the steps of expanding the first region to a first predetermined degree of the circumferential plastic strain and expanding the second region to a second predetermined degree of the circumferential strain, the second predetermined degree of the circumferential strain being different from the first predetermined degree of circumferential plastic strain.

Included in the drawing step is the step of orienting molecules comprising the polymer in a direction lengthwise along the billet. Similarly, the method further includes the step of orienting molecules comprising the polymer in a direction circumferentially around the lumen during the expanding step. The method may also include a step comprising implanting the billet within the vessel, the expansion step occurring after the implanting step.

The invention also includes a method of treating a stenosis in a vessel such as a coronary artery comprising the steps of:

(A) supplying an elongated billet formed of a polymer material, the billet having been drawn lengthwise to establish a predetermined degree of lengthwise plastic strain therein, a lumen extending lengthwise along the billet having been formed therein;

(B) positioning the billet within the vessel at the stenosis;

(C) heating the billet; and

(D) expanding the billet radially outwardly to open the stenosis.

### BRIEF DESCRIPTION OF THE DRAWINGS

FIG. 1 is a perspective view of a billet used to form a stent according to the invention;

FIG. 2 is a perspective view of a stent formed from the billet shown in FIG. 1;

FIG. 3 is a perspective view of another embodiment of a stent according to the invention;

FIG. 4 is a perspective view of yet another embodiment of a stent according to the invention;

FIG. 5 is a perspective view of a billet for forming a stent according to the invention;

FIG. 6 is a perspective view of a stent formed from the billet shown in FIG. 5;

FIG. 7 is a perspective view of another embodiment of a billet for forming a stent according to the invention;

FIG. 8 is a perspective view of a stent formed from the billet shown in FIG. 7;

FIG. 9 is a perspective view of a stent formed from a helically coiled filamentary member;

FIG. 10 is a perspective view of a stent having a helical molecular orientation; and

FIGS. 11-13 are longitudinal sectional views of a stent according to the invention being used to treat a stenosis in a vascular vessel.

### DETAILED DESCRIPTION OF THE EMBODIMENTS

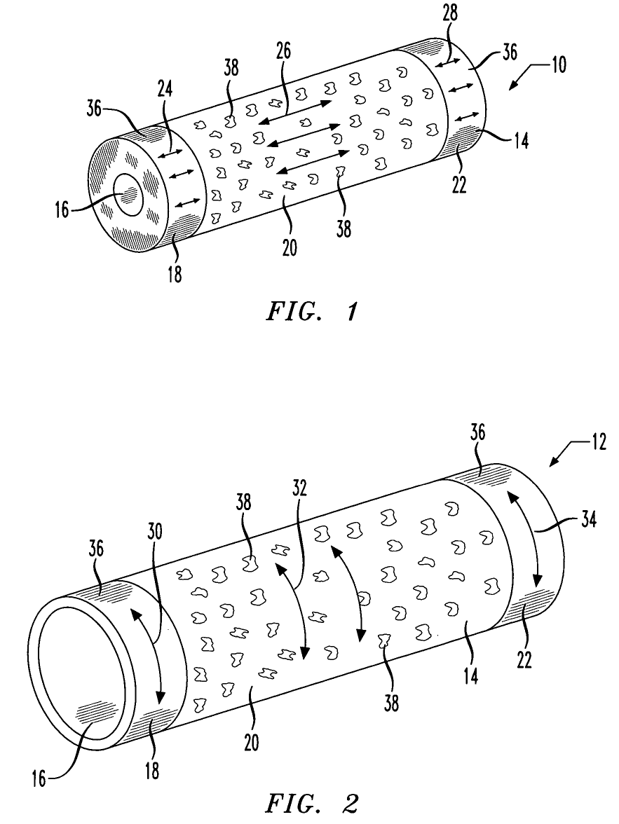

FIG. 1 illustrates a billet 10 which is expandable radially outwardly to form a stent 12 according to the invention, shown in FIG. 2. Billet 10 is comprised of a polymer material 14 and may be formed by extruding techniques as well as injection, compression or cast molding. A pre-drawn rod or polymer sheet material may also be used to form billet 10. A lumen 16 extends lengthwise through the billet, the lumen being formed during the extrusion process or drilled, for example, when the billet is a pre-drawn rod. Billet 10 is formed of one or more regions 18, 20, and 22 positioned lengthwise adjacent to one another in this example embodiment. The regions may be comprised of the same material or from different materials. The regions are further distinguished from one another by having differing degrees of plastic strain imparted by drawing the billet 10 lengthwise. The degrees of plastic strain are symbolized by the use of arrows 24, 26 and 28, the strain being oriented lengthwise along the billet commensurate with the direction of the draw. The molecule chains comprising the polymer material 14 are oriented along the length of the billet as a result of the drawing process. As described below, the plastic strain imparted by the drawing process is used to control the mechanical properties of the polymer material 14 comprising billet 10 to advantage.

The drawing process may be accomplished by drawing the billet 10 through dies of varying diameters smaller than the diameter of billet, the degree of strain being controlled by the size of the die and the speed and force used to draw the billet as well as the temperature of the billet during the draw. Differing regions may be created by drawing the different regions at different speeds and under different force and at different temperatures from one another and by attaching segments together end to end, the segments having been plastically deformed to different respective predetermined plastic strains. Other techniques, such as stretching the billet or the segments, may also be used to induce the desired degree of plastic strain. The segments are attached by adhesive bonding, heat fusion as well as other techniques.

The stent 12, shown in FIG. 2, is formed by expanding the billet radially outwardly about the lumen 16. The billet 10 is expanded when the stent 12 is being implanted within a vessel (described in detail below) during a surgical procedure, for example, to treat a coronary stenosis. The expansion of the billet 10 matches its diameter to the diameter of the vessel to open and support the vessel, thereby maintaining its patency. The radial expansion of billet 10 imparts further plastic strain to the polymer 14, the strain being oriented circumferentially around the lumen 16 as represented by the arrows 30, 32 and 34. The circumferential strains may be different from one another over the various regions 18, 20 and 22 due to the different lengthwise strains 24, 26 and 28 imparted to these regions on the billet 10. Molecule chains of the polymer material 14 are oriented in the circumferential direction as a result.

It is advantageous to heat the billet 10 to facilitate the radial expansion. The billet is preferably heated to a temperature between its glass transition temperature and its melting temperature and then allowed to cool while maintained in its expanded configuration. This fixes the diameter of the stent 12 to the desired size and mitigates elastic “spring back” of the stent to a smaller diameter, which is advantageous especially if the stent depends upon frictional forces between it and the vessel wall to hold the stent in place. Expansion and heating within the vessel is accomplished by means of a balloon catheter as described below. When the stent 12 is used to treat living tissue, such as a vascular stenosis, the temperature range must also be compatible with the tissue. The range of about 37° C. to about 70° C. is found to be useful in that it is between the glass transition and melting temperatures of various polymers and will not significantly adversely affect living tissue.

In forming the stent 12, billet 10 is drawn, heated and expanded in order to establish a desired stent diameter, as well as to establish a desired set of mechanical properties for each region of the stent. The lengthwise draw and the radial expansion as well as the temperature may be controlled, for example, to maximize the strength, stiffness, porosity or void content of various regions of the stent 12 as required for a particular application.

This capability is particularly advantageous when the stent 12 is used to administer medicaments or other compounds through the vascular system. One or more medicaments or compounds may be distributed throughout or coated onto one or more of the regions 18, 20 and 22, the medicament or compound being released into the blood stream as blood flows through the stent from voids or pores created within the stent 12 by the drawing and expansion processes.

The polymer 14 comprising billet 10 may be a bio-stable, bio-compatible material such as polyester, polypropylene, nylon polytetrafluoroethylene or other polymers with a history of success as implants in living tissue. Bio-absorbable polymers are also feasible. Such materials include polylactide, polyglycolide, polycaprolactone, tyrosine and their co-polymers.

Medicaments that may be administered from the stent include anti-inflammatory compounds such as Dexamethasone, anti-proliferates such as Rapamycin and Taxol, migration inhibitors such as Batimastat, as well as compounds to promote healing such as 17beta-estradiol. Anti-thrombogenic compounds such as heparin and phosphoricolyne are also feasible.

Inert compounds may be introduced into the billet 10 which increase the radiopacity of the stent 12, allowing it to be viewed using fluoroscopic techniques during and after implantation. When used with a billet 10 formed of bio-absorbable material, the radiopaque compounds comprise constituents that are not metabolized but are excreted or stored in the body. Radiopaque compounds are frequently metal powders having particle sizes on the order of 10 microns or less, allowing them to be safely released into the blood stream as the stent is absorbed without the danger of a stroke. Radiopaque materials include tantalum, zirconium, titanium, platinum, as well as barium compounds, bismuth compounds and compounds of iodine.

In a practical example associated with the embodiment exemplified by billet 10 and stent 12 of FIGS. 1 and 2, a radiopaque compound 36 is distributed throughout regions 18 and 22, which are also drawn and expanded to substantially maximize their strength to provide radial support to the stent. An anti-thrombogenic drug 38 is distributed throughout region 20, and this region is drawn and expanded so as to create voids in the region to facilitate release of the drug into the blood stream. When used to treat a vascular disorder, such as a coronary stenosis, the end regions 18 and 22 allow the stent 12 to be observed during and after the implant procedure and receive the bulk of the stresses imposed on the stent. The voids in region 20 provide for an initial release of the anti-thrombogenic drug to prevent any clots from forming downstream of the stent. Further drug release occurs as the stent degrades when it is comprised of a bio-absorbable material.

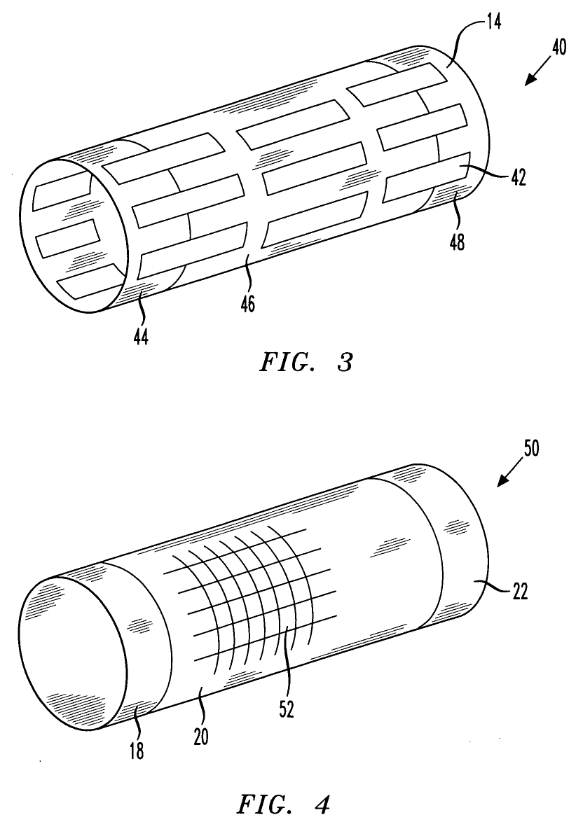

Other stent embodiments according to the invention are illustrated in FIGS. 3-6. As shown in FIG. 3, stent 40 has openings 42 formed therethrough. The stent also has regions 44, 46, and 48 of varying plastic strain imparted by drawing and expanding a billet to form the stent as described above. The openings may take virtually any shape and are formed in the billet by means such as laser cutting or traditional machining methods. The openings may be designed, for example, to promote the in growth of living tissue to the stent or to better control its flexibility.

In another embodiment, shown in FIG. 4, a stent embodiment 50 is formed from a plurality of interlaced filamentary members 52 formed from a polymer. The filamentary members are pre-drawn lengthwise to the desired degree of plastic strain before being interlaced to form the billet. Interlacing may be by means of weaving, knitting or braiding. Again, the stent 50 is formed from a pre-drawn billet which is expanded.

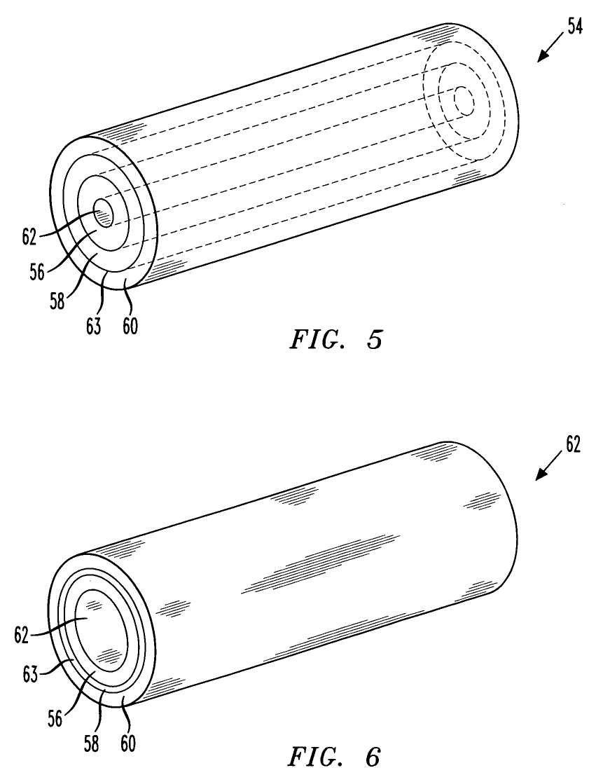

A billet 54 for yet another stent embodiment is shown in FIG. 5. Billet 54 comprises three regions 56, 58 and 60 having differing plastic strains imposed by lengthwise draws imposed on the regions. Region 56 is innermost and surrounds the lumen 62 extending lengthwise through the billet 54. Region 58 surrounds region 56 and region 60 surrounds region 58. Billet 54 may be constructed, for example, by first extruding cylinders of increasing inner diameter, drawing the cylinders to produce the desired amount of plastic strain, and then nesting the cylinders one within the other. The cylinders preferably fit closely within one another and may be adhesively bonded or heat fused together. The billet 54 is expanded radially as shown in FIG. 6, preferably using heat to produce the stent 62 having regions 56, 58 and 60 with differing mechanical properties located coaxially within one another. In addition to having differing mechanical properties, different medicaments or other compounds may be distributed throughout the various regions according to the invention. It is also possible to position a layer of radiopaque material 63 between the regions as shown in order to render the stent visible under fluoroscopic devices.

An example of a practical application of the stent 62, region 58 may be drawn and expanded so as to substantially maximize its strength, and form, within the stent 62, a robust shell which takes all of the stresses imposed on the stent and prevent its collapse. Outermost region 60 may be drawn and expanded to produce voids of a size which favor the ingrowth of living tissue to help hold the stent 62 in place within a vascular vessel. Innermost region 56 may contain an anti-thrombogenic drug and be drawn and expanded to substantially maximize release of the drug from the stent to mitigate clot formation and avoid a stroke as a result of the implantation of the stent.

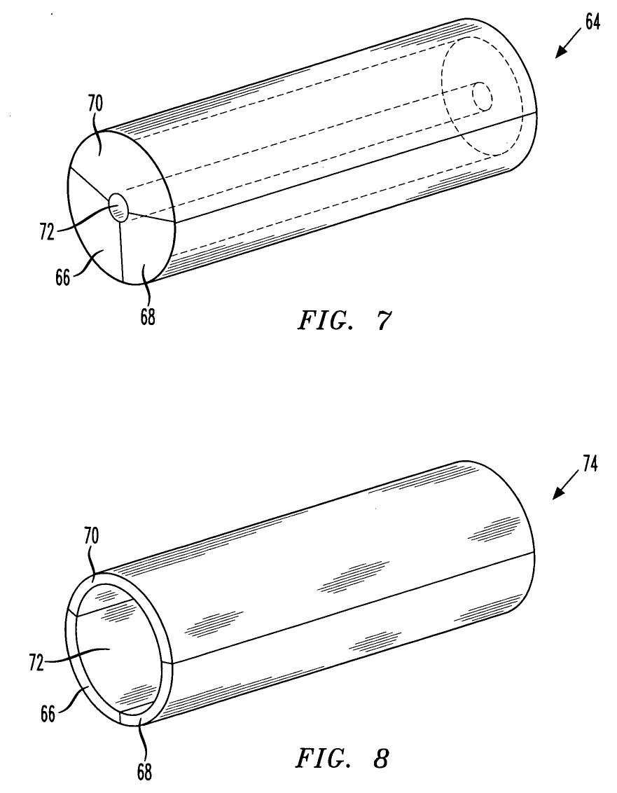

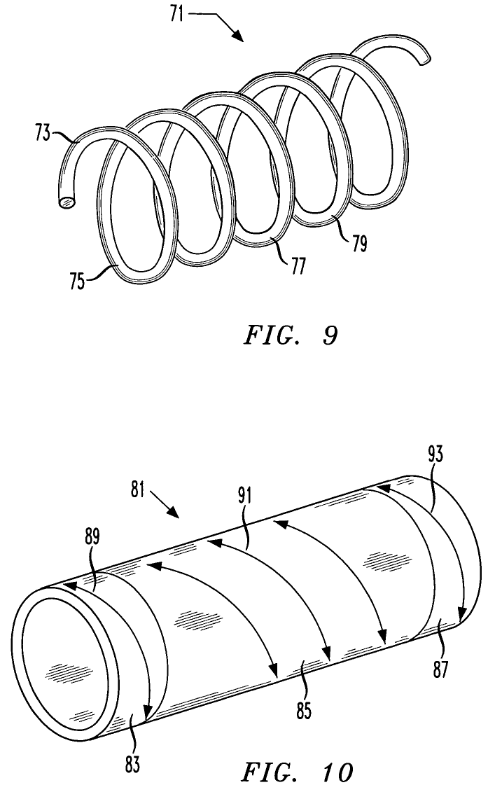

Another embodiment of a billet 64 is shown in FIG. 7. Billet 64 has pie shaped regions 66, 68 and 70 extending radially outwardly from the lumen 72. Such a billet is formed from drawn tubes cut into complementary shapes so that when assembled they will form the cylindrical billet 64, which may then be expanded to produce the stent embodiment 74 shown in FIG. 8, that has regions 66, 68 and 70 with differing properties over its surface.

In a further stent embodiment 71, shown in FIG. 9, the stent is formed from a helically coiled polymer filamentary member 73. Filamentary member 73 is pre-drawn lengthwise to a predetermined degree of plastic strain, and may also have different regions 75, 77, 79 for example having different degrees of plastic strain to impart different properties of strength, stiffness, and porosity as described above. Furthermore, the filamentary member 73 may be comprised of a bio-absorbable or bio-stable material and have additional compounds, such as medicaments and radiopaque marker compounds distributed throughout the various regions.

The billet (not shown) from which stent 71 is derived is formed by coiling the pre-drawn filamentary member 73 about a mandrel and using heat or chemical techniques to bias it into a helical shape having a smaller inside diameter than the stent 71. Expansion of the billet imparts further strain to finalize the configuration of the stent 71 and its properties.

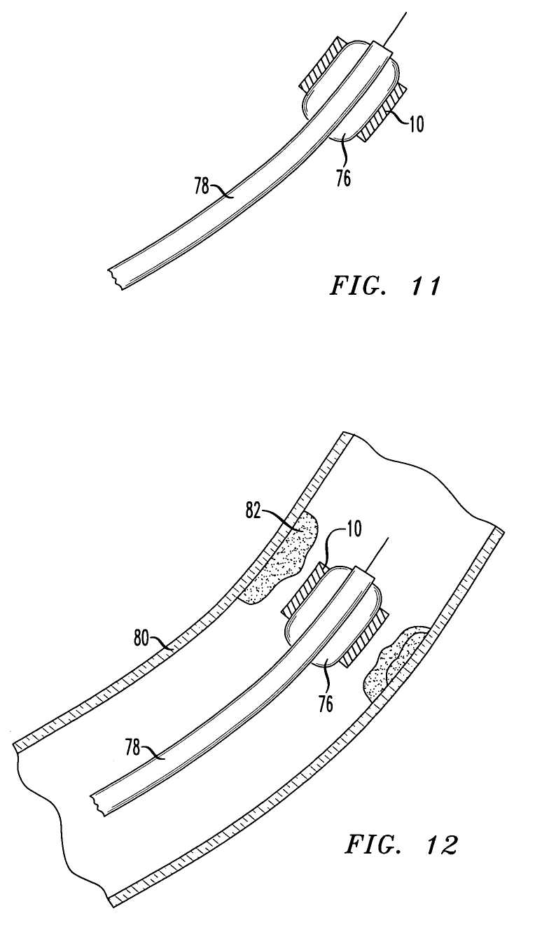

A stent which is expanded radially may have a molecular orientation that is not completely circumferential. The molecular orientation after the expansion depends on the molecular orientation of the billet, the temperature at which it is expanded, and the ratio of diameters of the billet to the stent before and after expansion. An example of a stent embodiment 81 not having a completely circumferential molecular orientation is shown in FIG. 10. The stent 81 has various regions 83, 85 and 87, each having a substantially helical molecular orientation as indicated by arrows 89, 91 and 93. The plastic strains as well as the orientation may be different across the different regions. The helical orientation is achieved, for example, by applying a twist to the billet as it is being drawn, or through the use of secondary processes such as compression molding after the drawing process.

Application of a billet and stent according to the invention to treat a vascular stenosis is illustrated in FIGS. 11-13. The billet and stent embodiments 10 and 12 are featured in this example, it being understood that any embodiment, either those described above or that otherwise falls within the scope of the invention may be used.

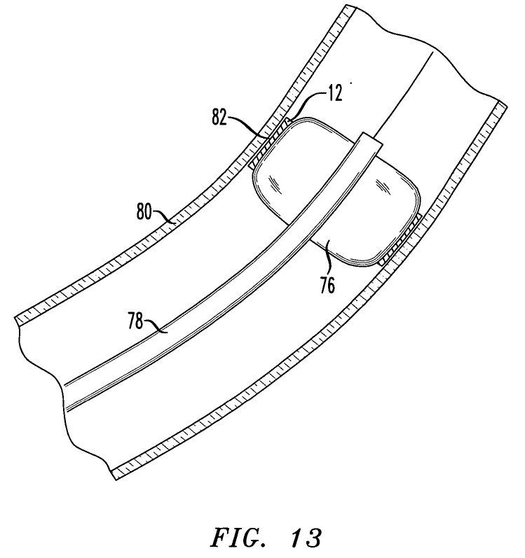

As shown in FIG. 11, billet 10 is positioned surrounding an inflatable balloon 76 at the end of a catheter 78. The balloon 76 is inflated slightly to retain the billet 10 to the catheter. As shown in FIG. 12, the catheter 78 is positioned within a vascular vessel 80 with the billet 10 at the position of the stenosis 82. A heated fluid is then pumped through the catheter and circulated through the balloon to heat the billet 10 to the desired temperature that will facilitate expansion of the balloon. Other methods are also feasible for heating the balloon and the billet including laser heating, radio frequency heating, resistive wires and vibrations, for example, ultrasonic waves.

Once the billet 10 is at the desired temperature, the balloon 76 is inflated as shown in FIG. 13. The inflated balloon expands the billet 10 to form the stent 12, the expansion imposing the circumferential strains and orienting the molecules comprising the polymer in order to establish the desired material properties of the stent. The stent 12 is forced into engagement with the vessel 80 and opens the stenosis. The balloon 76 and the stent 12 are then cooled or allowed to cool and the stent 12 remains in the expanded configuration. The balloon is then deflated and the catheter removed from the stent 12 and the vessel 80. If medicaments are distributed throughout the various regions of the stent, they are subsequently released into the blood stream.

Stents according to the invention formed from billets having regions of differing plastic strain imposed by drawing and expanding the billet forming the stent provide an efficient and effective means of treating vascular disorders, as well as delivering medicaments into the blood stream which will facilitate the treatment of the disorder.

The mechanical properties of the stent are readily controlled to provide a self-reinforced, strong, tough, expanded structure which may be either bio-absorbable or bio-stable.

What is claimed is:

1. A stent implantable within a vessel to support said vessel and ensure patency thereof, said stent comprising:
an elongated billet of polymer material, said billet being pre-drawn lengthwise and having a predetermined degree of lengthwise plastic strain; and
a lumen extending lengthwise through said billet.

2. A stent according to claim 1, wherein said billet is expanded radially outwardly from said lumen to a predetermined degree of circumferential plastic strain.

3. A stent according to claim 2, wherein said degree of said plastic strains are optimized to substantially maximize mechanical strength of said stent.

4. A stent according to claim 1, wherein said polymer comprises a bio-absorbable compound.

5. A stent according to claim 4, wherein said bio-absorbable compound is selected from the group consisting of polylactide, polyglycolide, polycaprolactone and tyrosine.

6. A stent according to claim 4, further comprising a medicament distributed throughout said polymer.

7. A stent according to claim 6, wherein said medicament is selected from the group consisting of Dexamethasone, Rapamycin, Taxol, Batimastat, 17beta-estradiol, heparin and phosphoricolyne.

8. A stent according to claim 6, wherein said degree of plastic strains are optimized to substantially maximize release of said medicament from said stent.

9. A stent according to claim 1, further comprising a radiopaque compound distributed throughout said polymer.

10. A stent according to claim 9, wherein said radiopaque compound is selected from the group consisting of tantalum, zirconium, titanium, platinum, compounds including barium, compounds including bismuth and compounds including iodine.

11. A stent according to claim 2, wherein said billet is heated during said radial expansion.

12. A stent according to claim 11, wherein said billet is heated to a temperature above the glass transition temperature of said polymer.

13. A stent according to claim 2, wherein said radial expansion is effected after said billet is positioned within said vessel.

14. A stent according to claim 1, wherein molecules comprising said polymer are oriented lengthwise along said billet in response to said lengthwise plastic strain.

15. A stent according to claim 2, wherein molecules comprising said polymer are oriented substantially circumferentially around said lumen in response to said circumferential plastic strain.

16. A stent according to claim 1, wherein molecules comprising said polymer forming said billet are oriented substantially helically around said lumen.

17. A stent according to claim 1, wherein molecules comprising said polymenr are oriented substantially helically around said lumen in response to said circumferential plastic strain.

18. A stent according to claim 1, wherein said billet comprises a molded body.

19. A stent according to claim 1, wherein said billet comprises a plurality of interlaced filamentary members.

20. A stent according to claim 19, wherein said filamentary members are interlaced by a technique selected from the group consisting of braiding, weaving and knitting.

21. A stent according to claim 1, wherein said billet comprises a single filamentary member biased into a helical shape.

22. A stent implantable within a vessel to support said vessel and ensure patency thereof, said stent comprising:
an elongated billet of polymer material, said billet having a first region, pre-drawn lengthwise to a first predetermined degree of lengthwise plastic strain, and a second region, pre-drawn lengthwise to a second predetermined degree of lengthwise plastic strain; and
a lumen extending lengthwise through said billet.

23. A stent according to claim 22, wherein said billet is expanded radially outwardly from said lumen, said first region having a first predetermined degree of circumferential plastic strain, said second region having a second predetermined degree of circumferential plastic strain.

24. A stent according to claim 22, wherein said plastic strains of one of said first and second regions are optimized to substantially maximize mechanical strength of said one region.

25. A stent according to claim 23, wherein said polymer comprises a bio-absorbable compound.

26. A stent according to claim 22, further comprising a first medicament distributed throughout said polymer comprising said first region.

27. A stent according to claim 26, where said degree of said plastic strains in said first region is optimized to substantially maximize release of said first medicament from said first region.

28. A stent according to claim 26, further comprising a second medicament different from said first medicament distributed throughout said polymer comprising said second region.

29. A stent according to claim 22, wherein a radiopaque marker is distributed throughout at least one of said first and second regions.

30. A stent according to claim 22, wherein said first region is comprised of a material different from said second region.

31. A stent according to claim 22, where said first and second regions are positioned adjacent to one another lengthwise along said billet.

32. A stent according to claim 22, wherein said first region is positioned between said lumen and said second region.

33. A stent according to claim 22, wherein said first and second regions are positioned circumferentially around said billet and extend radially outwardly from said lumen.

34. A stent according to claim 22, wherein said billet further comprises a third region, pre-drawn lengthwise to a third predetermined degree of lengthwise plastic strain, said third region having a third predetermined degree of circumferential plastic strain.

35. A stent according to claim 34, wherein said third predetermined degree of plastic expansion of said third region is different from said degree of plastic expansion of both said first and second regions.

36. A stent according to claim 34, wherein said third region further comprises a third compound distributed throughout said polymer comprising said third region.

37. A stent according to claim 36, wherein said third compound comprises a medicament.

38. A stent according to claim 36, wherein said third compound comprises a radiopaque marker.

39. A stent according to claim 35, wherein said first, second and third regions are positioned adjacent to one another lengthwise along said billet with said second region positioned between said first and third regions.

40. A stent according to claim 35, wherein said first, second and third regions are positioned in overlying relation circumferentially around said billet substantially surrounding said lumen, said second region being positioned between said first and third regions.

41. A stent according to claim 35, wherein said first, second and third regions are positioned circumferentially around said billet and extend radially outwardly from said lumen.

42. A method of making a stent implantable within a vessel to support said vessel and ensure patency thereof, said method comprising the steps of:
supplying an elongated billet formed of a polymer material;
drawing said billet lengthwise to establish a predetermined degree of lengthwise plastic strain; and
forming a lumen extending lengthwise along said billet.

43. A method according to claim 42, further comprising the step of expanding said billet radially outwardly from said lumen to establish a predetermined degree of circumferential plastic strain therein.

44. A method according to claim 43, further comprising the step of heating said billet during said radial expansion step.

45. A method according to claim 44, wherein said billet is heated to a temperature above the glass transition temperature of said polymer material.

46. A method according to claim 42, wherein said drawing step further comprises the steps of drawing a first region of said billet to a first predetermined degree of said lengthwise plastic strain, and drawing a second region of said billet to a second predetermined degree of said lengthwise plastic strain, said second predetermined degree of said lengthwise plastic strain being different from said first predetermined degree of lengthwise plastic strain.

47. A method according to claim 43, wherein said expanding step further comprises the steps of radially expanding said first region to a first predetermined degree of said circumferential plastic strain and expanding said second region to a second predetermined degree of said circumferential strain, said second predetermined degree of said circumferential strain being different from said first predetermined degree of circumferential plastic strain.

48. A method according to claim 43, further comprising the step of implanting said billet within said vessel, said expansion step occurring after said implanting step.

49. A method of treating a stenosis in a vessel, said method comprising the steps of:
supplying an elongated billet formed of a polymer material, said billet having been drawn lengthwise to establish a predetermined degree of lengthwise plastic strain therein, a lumen extending lengthwise along said billet having been formed therein;
positioning said billet within said vessel at said stenosis;
heating said billet; and
expanding said billet radially outwardly to open said stenosis.
